(19) United States
(12) Patent Application Publication
(10) Pub. No.: US 2004/0067510 A1
(43) Pub. Date: Apr. 8, 2004

(54) Model of transmissible spongiform encephalopathic (tse) disease

(76) Inventor: Toby James Gibson, Heidelberg (DE)

Correspondence Address: WENDEROTH, LIND & PONACK, L.L.P., 2033 K STREET N. W., SUITE 800, WASHINGTON, DC, 20006-1021, US

(21) Appl. No.: 10/468,484

(22) PCT Filed: Feb. 20, 2002
(86) PCT No.: PCT/GB02/00731

(30) Foreign Application Priority Data
Feb. 20, 2001 (GB) 0104168.0

(51) Int. Cl. C12Q 1/68

(52) U.S. Cl. 435/6

(57) ABSTRACT

The invention relates to the mechanism of transmission of spongiform encephalopathics. The invention provides simple diagnostic tests for animals that are infected with transmissible spongiform encephalopathic (TSF.) disease. The test comprises testing an animal for the presence of mutant non-viral retroelement nucleic acid molecules, wherein the presence of said mutant retroelement nucleic acid molecules is indicative of infection. The invention also provides methods for the treatment of TSE disease by administering to an animal a therapeutically-effective amount of a compound that is effective to conteract the effect of a non-viral mutant retroelement nucleic acid molecule in inducing TSE disease.

The invention relates to the mechanism of transmission of spongiform encephalopathies The invention provides simple diagnostic tests for animals that are infected with transmissible spongiform encephalopathic (TSE) disease. The invention also provides methods for the treatment of TSE disease.

Despite decades of research, the agent responsible for transmitting spongiform encephalopathies (TSEs) has not yet been identified. TSE diseases include Creutzfeldt-Jakob disease (CJD), new variant Creutzfeldt-Jakob disease (vCJD), and Kuru, fatal familial insomnia (FFI) and Gestmann-Straussler syndrome (GSS) in humans, scrapie in sheep and bovine spongiform encephalopathy (BSE). These diseases may arise sporadically, as is typical of CJD, but are then transmissible by injection or ingestion of infected material (reviewed in Chesebro, B. (1999) Neuron 24, 503-506; Manson, J. C. (1999) Trends Microbiol. 7, 465467). The diseases differ in host range, with most TSE diseases having a restricted species range. However, other TSE diseases, most notably BSE, exhibit a very wide host range.

The Prion protein model dominates this field, and is based on the premise that modified host PrP protein acts as the transmissible disease-causing agent This model fits the observation that TSE diseases elicit almost no immune reaction in the host. Prion transmission has not been verified, however, as it has not been possible to produce pure PrP aggregates.

One long-standing objection to the Prion model is the observation that TSE diseases show classical genetic behaviours, such as reproducible strain variation, while also responding to selection for strain traits and showing adaptation to new hosts. Moreover, evidence has been steadily accumulating that infectious titre is decoupled from the quantity (or even the presence) of PrP deposits.

As a consequence of the Prion transmission model (Pusiner, S. B. (1991) Science 252, 1515-1522), most studies aimed at understanding TSEs have focussed on the PrP GPI-linked cell surface protein. This focus has yielded important benefits, and it is known from transgenic mouse studies that simple overexpression of PrP leads to PrP deposits which cause spongiform disease (Hsiao K. K. er al. (1990) Science 250, 1587-1590; Hegde, R. S. et al. (1998) Science 279, 827-834). Numerous polymorphisms in the PrP protein sequence ate also known to influence TSE disease progression strongly (see, for example, Furukawa. H., Kitamoto, T., Tanaka, Y. and Tateishi, J. (1995) Brain Res. Mol. Brain Res. 30, 385-388; Goldfarb, L. G., Brown, P., Cervenakova, L. and Gajdusek, D. C. (1994) Mol. Neurobiol. 8, 89-97), while knockout mice devoid of PrP are resistant to the consequences of TSE infection (Bueler, H. et al. (1993) Cell 73, 1339-1347), although they can harbour and pass on the transmissible agent (Race, R. and Chesebro, B. (1998) Nature, 392, 770). It is therefore fairly clear that PrP protein deposits are a direct cause of brain damage in TSEs.

By analogy with recent results in Alzheimer's disease (Schenk, D. et al. (1999) Nature 400, 173-177), immunisation with PrP deposits may enable the immune system to clear the deposits, so hindering disease progression. With regard to TSE transmissibility, however, results have been highly inconsistent, with several groups reporting that infectious titre is not correlated with quantity of PrP deposition (Hsiao, K. K. et al. (1994) Proc. Natl. Acad. Sci. U. S. A. 91, 9126-9130; Lasmezas, C. I. et al. (1997) Science 275, 402-405; Manson, J. C. et al., (1999) EMBO J., 18, 6855-6864). Most disquietingly, it has been shown that apparently resistant hosts can replicate infectious TSE agent, without any symptoms developing in the host animal (Race, R. and Chesebro, B. (1998) Nature, 392, 770; Hill, A. F. et al. (2000) Proc. Natl. Acad. Sci. U.S.A. 97, 10248-10253) and that perfectly healthy humans harbour an endemic agent that induces CJD in hamsters (Manuelidis, E. E. and Manuelidis, L. (1993) Proc. Natl. Acad. Sci. U.S.A. 90, 7724-7728).

There is currently an urgent need for a fast test for BSE infection in European Cattle. This is the only way reliably to prevent future infection of humans as well as continued infection of cattle. Maternal transmission may otherwise prevent eradication of BSE for many generations. Presently available diagnostic tests are largely ineffective, since these tests can only distinguish infected animals at an advanced stage of disease. By that point, not only is therapeutic intervention impossible, but transmission of the infective agent has often already occurred.

These tests will need to be performed continuously for a number of years to monitor and complete the eradication process. Furthermore, it now appears unlikely that BSE has been confined to Europe, meaning that other cattle populations around the world will now require testing to ensure that they are clear of disease.

Certain domesticated animals, such as pigs, are more closely related to cows than are humans or mice, both of which can suffer BSE infection. It is therefore thought that infection of pigs, poultry and other farm animals remains a theoretical worry and will need extensive testing, even in the hoped for case that no transmission is found. At present, there is no test for TSE disease in these animals.

There is also a need for tests for all other forms of TSEs in domestic animals, such as scrapie in sheep, feline spongiform encephalopathy in cats, transmissible mink encephalopathy, and, potentially, chronic wasting disease (CWD) in undomesticated mule deer and elk. While scrapie is a less urgent problem than BSE, large economic benefits would follow for farmers from its eradication in the United Kingdom flock and from flocks in other scrapie-infected countries. An early, sensitive and reliable test to monitor scrapie would allow its complete eradication and the certification of scrapie-free flocks.

As well as testing living animals in order to eradicate BSE, tests of all cattle (and other) carcasses that are destined for the human food industry would no doubt be obligatory for some years, until the diseases were regarded as eradicated. Additionally, there are a huge number of products that contain bovine materials, including materials as diverse as cosmetic lipsticks, and vaccines containing bovine serum. Further concerns have been raised about baby food, baby milk powder, and the milk substitute that is fed to bovine calves. The source material for all these materials must be uninfected.

There is also a need for a test to confirm diagnosis of suspected cases of vCJD in humans, as well as tests for traditional CJD and GSS.

It is the Applicant's contention that the agent that is responsible for transmissible spongiform encephalopathies has not yet been identified. The identification of the correct agent would allow the design of diagnostic tests to distinguish infected animals, as well as allowing the development of therapeutic strategies that are able to counter these diseases.

According to the invention, there is provided a method for diagnosing an animal for a transmissible spongiform encephalopathy disease, said method comprising testing the animal for the presence of mutant non-viral retroelement nucleic acid molecules wherein the presence of said mutant non-viral retroelement nucleic acid molecules is indicative of infection.

The method relies on a new mechanism proposed for TSE infection and propagation, whereby mutant retroelements are the transmissible agents for TSEs, while PrP protein deposition is the chief effector of pathogenicity. The model predicts that transfer of a TSE between species will lead to copies of a repetitive element present in the infected source material being multiply inserted into the genomes of newly infected cells and then being expressed as cellular RNA.

The model involves uncontrolled proliferation of retroelements, such as small dispersed repeat sequences (SINEs), in somatic cells. This proliferation induces overexpression of PrP, with pathogenic consequences. The mechanism involves twin tandem positive feedback loops, where triggering the second loop leads to the pathogenic disease. This model is consistent with the long latency period and much shorter visible disease progression that is typical of TSEs.

The proposed infectious agent satisfies the requirements that have been experimentally determined so far for the infectious agent of TSE diseases. For example, the infectious agent should be capable of being replicated in somatic cells of mammals, should not elicit an immune reaction, and must be relatively small (the smallest known viral genome or smaller), to fit the kinetics of irradiation inactivation that have been experimentally-determined for TSEs (Alper, T. et al. (1966) Biochem Biophys. Res. Commun. 22, 278-284; Rohwer, R. G. (1984) Nature 308, 658-662; Rohwer, R. G. (1991) Corr. Top. Microbiol. Immunol. 172, 195-232). Retroelement molecules fit all of these constraints.

There are a number of nonviral retroelement varieties that are presently known to reside in animal genomes, and the method of the invention includes testing any one of these retroelement varieties. Examples include short interspersed nuclear elements (SINEs), such as the human Alu sequences (reviewed in Jurka, J. (1998) Curr. Opin. Struct Biol. 8, 333-337; Schmid, C. W. (1998) Nucleic Acids Res. 26, 4541-4550; Smit, A. F. (1999) Curr. Opin. Genet. Dev. 9, 657-663), and long interspersed nuclear elements (LINEs) (Moran, J. V. (1999) Genetica., 107, 39-51). SINES are favoured as candidate infectious agents in the present model because they are the smallest retroelements that are presently known. Furthermore, they cannot elicit an immune response as they encode no protein, being solely composed of DNA genes and RNA transcripts. It is notable that SINES cannot replicate themselves, being dependent on reverse transcriptase and endonuclease proteins encoded in larger LYE retroelements LINEs are somewhat larger than SINEs, encoding their own replication system However, the encoded proteins are probably not highly expressed, meaning that these agents may manage to escape immune surveillance.

Over 40% of the human genome is derived from repetitive elements, including Alus (Smit, A. F. (1999) Curr. Opin. Genet: Dev. 9, 657-663) It is estimated that there are about 1,000,000 Alus per haploid human genome (Schmid, C. W. (1998) Nucleic Acids Res. 26, 4541-4550). Alu sequences are partially related to the 7S RNA of the signal recognition particle SRP. SINEs in primates and rodents generally are derived from 7S, whereas in most mammals, including domestic ungulates, SINEs exhibit a higher degree of sequence homology with tRNAs (Schmid, C. W. (1998) Nucleic Acids Res 26, 4541-4550). SINEs proliferate in the genome via retrotransposition using reverse transcriptase and endonuclease activities encoded; by larger LIKE family repetitive elements (Ohshima, K. et al. (1996) Mol. Cell. Biol. 16, 3756-3764).

SINES possess internal RNA Pol. III promoters, so that newly inserted genes may be immediately transcribed, though the chromosomal context will influence this. SINEs are primarily parasitic in nature, although as they have costed with their hosts for hundreds of millions of years, they may have coevolved on occasion to play useful roles in host cells (Schmid, C. W. (1998) Nucleic Acids Res. 26, 4541-4550).

In normal cells, SINE genes are expressed at low levels, with high turnover of RNAs, so that transcripts are in low abundance (Schmid, C. W. (1998) Nucleic Acids Res. 26, 4541-4550). To be transmitted in the host genome, retrotransposition of repeats must occur in the germline or in early embryogenesis. However, since the replication cycle of SINEs is decoupled from cellular genome replication, individual SINEs are under positive selection to spread through the cellular genome and somatic insertion also occurs (Economou-Pachnis, A. and Tsichlis, P. N. (1985) Nucleic Acids Res. 13, 8379-8387). Within any particular cell, “improved fitness” for a SE will be more efficient replication and therefore mutations that improve steps such as transcription, mRNA stability, priming for reverse transcription or integration into the genome will be selected. There will, of course, be counter-selection at the host organismal level, suppressing variants that affect its health and reproduction.

Two characteristics of SINEs are considered especially noteworthy with respect to TSE diseases. Firstly, when cells are stressed (e.g. by heat shock or viral infection), SINE gene expression increases dramatically and SINE RNAs may be orders of magnitude more abundant (Fornace, A. J. Jr. and Mitchell, J. B. (1986) Nucleic Acids Res. 14, 5793-5811; Panning, B. and Smiley, J. R. (1993) Mol. Cell. Biol. 13, 3231-3244; Liu, W. M. et al. (1995) Nucleic Acids Res. 23, 1758-1765). Secondly, SINE RNAs directly affect protein synthesis by binding and inhibiting PKR (eIF2 kinase), the dsRNA-activated kinase (Chu, W. M. et al. (1998) Mol. Cell. Biol. 18, 58-68). PKR is involved in cellular antiviral defence, the activated kinase shutting down cellular translation in the presence of long dsRNAs (Williams, B. R. (1999) Oncogene 18, 6112-6120). By inhibiting PKR, high concentrations of SINE RNAs cause concomitant increases in protein synthesis (Chu, W. M. et al. (1998) Mol. Cell. Biol. 18, 58-68; Schmid, C. W. (1998) Nucleic Acids Res. 26, 4541-4550).

FIG. 1 herein shows a scheme wherein two intersecting positive feedback loops would allow retroelements such as SINEs to operate as the causative agent in TSEs. In the first latent cycle, SINEs are iteratively selected for more efficient replication. New mutations arise during error-prone reverse transcription (and at a lower rate during RNA Pol. III transcription). Proliferation of replication-competent SINE genes results in increased SINE RNA concentration. Since initial RNA concentrations are low and SINE retrotransposition events are rare, several rounds of improvement will need to be completed and the latent period will be long (normally exceeding the lifetime of the organism). Eventually, the SINE RNA concentration will increase sufficiently such that it begins to shut down cellular PKR activity, leading to generally increased protein synthesis. Ibis engages the second, much more virulent pathogenic cycle into operation. Increased protein synthesis raises PrP production to the point at which PrP aggregates begin to form between cells. Cells become stressed by the deposits, leading to a massive increase in SINE gene transcription, further feeding the cycle.

This process could occur in any cell type, but will particularly affect long-lived terminally differentiated cell types such as neurones, just as is seen with TSE diseases. To be infectious, the SINE nucleic acid, either as RNA, DNA or both, must be readily transferred between cells (although the process does not need to achieve virus-like efficiency). The mechanism of the transfer mechanism is presently unclear, although it may involve, for example, endocytic uptake of SINE RNA released by lysed cells, piggyback in endogenous retrovirus particles, or vesicle or cell fusion Antisense oligonucleotides are known to be taken up by animal cells by endocytosis, albeit inefficiently Moreover, in genetic interference experiments, double-stranded RNAi has been shown to spread throughout the worm Caenorhabditis elegans if it is injected into the body cavity (Fire, A. et al. (1998) Nature 391, 806-811), if the worm is bathed in dsRNA preparations (Tabara, H., Grishok, A. and Mello, C. C. (1998) Science 282, 430-431), or even if the dsRNA is expressed in Escherichia coli, then ingested (Timmons, L and Fire, A. (1998) Nature 395, 854). According to the method of the invention, the diagnostic method may comprise the steps of:

a) contacting a sample of tissue from the animal with a nucleic acid probe under stringent conditions that allow the formation of a hybrid complex between the mutant non-viral retroelement nucleic acid molecule and the probe;

b) contacting a control sample with said probe under the same conditions used in step a); and

c) detecting the presence of hybrid complexes in said samples; wherein detection of levels of the hybrid complex in the animal sample that differ from levels of the hybrid complex in the control sample is indicative of the TSE disease.

The term “hybridisation” used herein refers to any process by which a strand of nucleic acid binds with a complementary strand of nucleic acid by hydrogen bonding, typically forming Watson-Crick base pairs. As carried out in vitro, one of the nucleic acid populations is usually immobilised to a surface, whilst the other population is free. The two molecule types are then placed together under conditions conducive to binding.

Stringency of hybridisation refers to the percentage of complementarity that is needed for duplex formation. “Stringency” thus refers to the conditions in a hybridization reaction that favour the association of very similar molecules over association of molecules that differ. Conditions can therefore exist that allow not only nucleic acid strands with 99-100% complementarity to hybridise, but sequences with lower complementarity (for example, 50%) to also hybridise Standard stringent DNA-DNA hybridisation conditions are defined herein as overnight incubationat 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. (see Sambrook and Russell, Molecular Cloning, A Laboratory Manual (2000) Cold Harbor-Laboratory Press, Cold Spring Harbor, N.Y. or Ausubel er al., Current protocols in molecular biology (1990) John Wiley and Sons, N.Y.).

Numerous techniques now exist for effecting hybridisation of nucleic acid molecules. Such techniques usually involve one of the nucleic acid populations being labelled. Labelling methods include, but are not limited to, radiolabelling, fluorescence labelling, chemiluminescent or chromogenic labelling, or chemically coupling a modified reporter molecule to a nucleotide precursor such as the biotin-streptavidin system. This can be done by oligolabelling, nick-translation, end-labelling or PCR amplification using a labelled polynucleotide. Labelling of RNA molecules can be achieved by cloning the DNA sequences encoding the RNA transcript of the invention into a vector specifically for this purpose. Such vectors are known in the art and may be used to synthesise RNA probes in vitro by the addition of an appropriate RNA polymerase such as T7, T3 or SP6 and labelled nucleotides.

Various kits are commercially available that allow the labelling of molecules (for example, Pharmacia & Upjohn (Kalamazoo, Miss.); Promega (Madison Wis.); and the U.S. Biochemical Corp. (Cleveland, Ohio.). Hybridisation assays include, but are not limited to dot-blots, Southern blotting, Northern blotting, chromosome in situ hybridisation (for example, FISH [fluorescence in situ hybridisation]), tissue in situ hybridisation, colony blots, plaque lifts, gridded clone hybridisation assays. DNA microarrays and oligonucleotide microarrays (see, for example, WO95/11995; Lockhart, D. J. et al. (1996) Nat. Biotech. 14: 1675-1680); and Schena, M. et al. (1996) PNAS 93: 10614-10619; W095/251116).

The invention therefore also embodies a diagnostic method involving detecting a non-viral mutant retroelement nucleic acid molecule, the method comprising the steps of: (a) contacting a nucleic acid probe with a biological sample under hybridising conditions to form duplexes: and (b) detecting any such duplexes that are formed. The term “probe” as used herein refers to a nucleic acid molecule in a hybridisation reaction whose molecular identity is known and is designed specifically to identify a specific nucleic acid species. Usually, the probe population is the labelled population, but this is not always the case, as for example, in a reverse hybridisation assay.

A further example of a suitable diagnostic method may comprise the steps of:

a) contacting a sample of nucleic acid from tissue of the animal with a nucleic acid primer under stringent conditions that allow the formation of a hybrid complex between the non-viral retroelement nucleic acid molecule and the primer;

b) contacting a control sample with said primer under the same conditions used in step a);

c) amplifying the sampled nucleic acid; and

d) detecting amplified nucleic acid from both patient and control samples; wherein detection of the amplified nucleic acid in the animal sample that differs significantly from the amplified nucleic acid in the control sample is indicative of TSE disease.

In one embodiment of this aspect of the invention, step d) of the method may comprise detecting the level of amplified nucleic acid from both patient and control samples. Detection of levels of the amplified nucleic acid in the animal sample that differ significantly from levels of the amplified nucleic acid in the control sample is indicative of TSE disease.

In an alternative, preferred, embodiment of this aspect of the invention, step d) of the method may comprise the step of sequencing the amplified nucleic acid from both patient and control samples, wherein detection of amplified nucleic acid in the animal sample of a different sequence to that of the amplified nucleic acid in the control sample is indicative of TSE disease. The amplified nucleic acid molecule may be sequenced directly, or it may be cloned for subsequent analysis. Methods for the analysis of the sequence of an amplified nucleic acid molecule are well known to the skilled reader; details of suitable methods may be found, for example, in Sambrook and Russell, Molecular Cloning, A Laboratory Manual (2000) Cold Harbor Laboratory Press, Cold Spring Harbor, N.Y. or Ausubel et al., Current protocols in molecular biology (1990) John Wiley and Sons, N.Y.

In a still further aspect of the method of the invention, the testing step may comprise the steps of:

a) obtaining a tissue sample from an animal being tested for TSE disease;

b) isolating nucleic acid from said tissue sample; and

c) diagnosing the animal for disease by detecting the presence of mutant non-viral retroelement nucleic acid molecule as an indication of the TSE disease.

As discussed above, this method may also comprise the step of sequencing the nucleic acid molecule isolated from the tissue sample, in order to diagnose TSE disease.

Suitable tissues for testing for TSE disease include body fluids such as blood, peritoneal fluid, urine and saliva, and also solid tissues, such as, for example, brain tissue. One useful result of the invention is the ability to test transplant tissues for the presence of TSE disease, to ensure that the transplant tissue is not infectious. Such tissues may include any tissue that is transplanted into a human or animal patient, including corneal transplants.

Any one of the methods discussed in detail above may be carried out in vitro.

According to a further aspect of the present invention, there is provided a method of treating a TSE disease in an animal in need of such treatment by administering to an animal a compound that is effective to counteract the effect of a non-viral mutant retroelement nucleic acid molecule in inducing TSE disease, in a therapeutically effective amount.

A variety of such compounds are likely to exist. For example, a suitable compound may comprise an RNAi molecule that is targeted to the non-viral mutant retroelement nucleic acid molecule, thus utilising the phenomenon of gene silencing that is known to occur in many eukaryotes in the presence of dsRNA that exhibits a high degree of homology with a target gene. In this manner, it is envisaged that it may be possible to use RNAi to silence rogue retroelements, so terminating the proliferative cycle discussed above. Of course, this approach will require caution, since it is possible that retroelements such as SINEs also have some positive effects on the host genome.

One embodiment of this aspect of the invention involves a different strategy, wherein a modified PKR kinase molecule whose activity is not inhibited by a non-viral mutant retroelement nucleic acid molecule is used to counteract the effect of the non-viral mutant retroelement nucleic acid molecule in causing TSE disease. The PKR kinase is a very powerful regulator of translation that is central to antiviral defence, among other functions. By adding further copies of a modified PKR molecule, or of a gene encoding a modified PKR molecule, it is envisaged that translation may be down-regulated in infected cells. This will lead to reduced PrP expression and prevention of PrP deposits, so preventing the disease pathology.

Modified PKR mutants whose activity is not inhibited by a non-viral mutant retroelement nucleic acid molecule are not presently known. However, it is envisaged that it will be possible to isolate or engineer PKR variants, including natural biological variants and mutants (such as mutants containing amino acid substitutions, insertions or deletions) of the wild type sequence, that possess the desired properties. It is considered that such an exercise is within the abilities of the person of skill in the art.

In order that a compound effective to counteract the effect of a non-viral mutant retroelement nucleic acid molecule in inducing TSE disease is internalised into a diseased cell, one effective route of administration may be to provide a nucleic acid molecule encoding the compound directly to tile animal in an expressible vector, such as a vector comprising expression control sequences operably linked to the nucleic acid molecule (gene therapy). Gene therapy may thus be used to replace the normal wild type PKR gene with an engineered gene that encodes a modified PKR protein. Using selective targeting strategies, gene therapy may be localised to specific cell types or tissues to ensure that the normal function of the PKR kinase is not unduly compromised.

Treatment by gene therapy may be effected either in vivo or ex vivo. Ex vivo gene therapy generally involves the isolation and purification of the animal cells, introduction of the therapeutic gene into the cells and finally, the introduction of the genetically-altered cells back into the animal. In vivo gene therapy does not require the isolation and purification of cells prior to the introduction of the therapeutic gene into the animal. Instead, the therapeutic gene can be packaged for delivery. Gene delivery vehicles for in vivo gene therapy include, but are not limited to, non-viral vehicles such as liposomes, replication-deficient viruses (for example, adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479). Alternatively, “naked DNA” may be directly injected into the bloodstream or muscle tissue as a form of in vivo gene therapy.

An alternative strategy to gene: therapy may use direct delivery of the PKR protein to the patient (for example, see Shwarze, S. R. et al. (1999) Science, 285, 1569-1572; Bayley, H. (1999) Nature Biotech., 17, 1666-1067). A therapeutically-effective amount of the PKR protein may be administered to the patient, optionally in conjunction with a pharmaceutically-acceptable carrier, such as a protein, polysaccharide, polylactic acid, polyglycolic acid or inactive virus particle. Carriers may also include pharmaceutically acceptable salts, liquids (such as water, saline, glycerol, ethanol) and/or auxiliary substances such as wetting or :emulsifying agents, and pH buffering substances. Carriers may enable the pharmaceutical compositions to be formulated into tablets, pills, dragees, capsules, liquids, gels, syrups, slurries, suspensions to aid intake by the patient. A thorough discussion of pharmaceutically acceptable carriers is available in Remington's Pharmaceutical Sciences (Mack Pub. Co., N.J. 1991).

One advantage of the direct delivery strategy over the gene therapy strategy discussed above is that delivery of the protein will be reversible, whereas using gene therapy, this is not generally the case.

The phrase “therapeutically effective amounts” used herein refers to the amount of PKR protein that is needed to treat, or ameliorate the TSE disease. An effective initial method to determine a “therapeutically effective amount” may be by carrying out cell culture assays or using animal models. In addition to determining the appropriate concentration range for the PKR agent to be therapeutically effective, animal models may also yield other relevant information such as preferable routes of administration (for example, enteral, intra-arterial, intrathecal, intramedullary, intramuscular, intranasal, intraperitoneal, intravaginal, intravenous, intraventricular, oral, rectal, subcutaneous, sublingual, transcutaneous or transdermal means).

Factors that may be taken into consideration when determining dosage include the severity of the disease state in the patient, the general health of the patient, the age, weight, gender, diet, time and frequency of administration, drug combinations, reaction sensitivities and the patient's tolerance or response to the therapy. The precise amount can be determined by routine experimentation but will ultimately lie with the judgement of the clinician. Generally, an effective dose will be from 0.01 mg/kg (mass of drug compared to mass of patient) to 50 mg/kg, preferably 0.05 mg/kg to 10 mg/kg. Compositions may be administered individually to a patient or may be administered in combination with other agents, drugs or hormones.

Typically, the therapeutic compositions may be prepared as injectables, either as liquid solutions or suspensions; solid forms suitable for solution in, or suspension in, liquid vehicles prior to injection may also be prepared. Direct delivery of the compositions can generally be accomplished by injection, subcutaneously, intraperitoneally, intravenously or intramuscularly, or delivered to the interstitial space of a tissue. Dosage treatment may be a single dose schedule or a multiple dose schedule. The invention also provides a method for monitoring the therapeutic treatment of a TSE disease in an animal, comprising monitoring over a period of time the level of expression of a non-viral mutant retroelement nucleic acid molecule in tissue from said patient, wherein altering said level of expression over the period of time towards a control level is indicative of regression of said disease. Animals for testing and treatment according to the invention include any animal that is subject to infection by a TSE disease. Preferably, the animal is a mammal, such as a human, or a domestic ungulate.

According to a still further aspect, the invention provides a non-viral mutant retroelement nucleic acid molecule that is implicated in the cause or progression of a TSE disease. Such a nucleic acid molecule is preferably a mutated SINE, or a mutated LINE nucleic acid molecule.

According to a still further aspect of the invention, there is provided a method for the identification of a compound that is effective in the treatment and/or diagnosis of a TSE disease, comprising contacting a non-viral mutant retroelement nucleic acid molecule with one or more compounds suspected of possessing affinity for said non-viral mutant retroelement nucleic acid molecule, and selecting a compound that binds specifically to said non-viral mutant retroelement nucleic acid molecule.

The invention also includes compounds that are identifiable by a method as described above. Examples of suitable such compounds may be nucleic acid molecules, enzymes, small organic molecules, peptidomimetics, inorganic molecules, peptides, polypeptides or antibodies. Suitable nucleic acid molecule compounds include RNAi molecules that are targeted to the non-viral mutant retroelement nucleic acid molecule that is implicated in TSE disease, as is discussed above. An example of a polypeptide compound according to this aspect of the invention is a modified PKR molecule, that is not inhibited by non-viral mutant retroelement nucleic acid molecules. Other examples will be apparent to the skilled worker, including, for example, compounds that interact with PKR kinase to prevent it being inhibited by the causative non-viral retroelement nucleic acid molecule, yet which allow the normal cellular function of the PKR kinase to remain uncompromised.

According to a still further aspect of the invention, there is provided a kit useful for diagnosing TSE disease comprising a first container containing a nucleic acid probe that hybridises under stringent conditions with a non-viral mutant retroelement nucleic acid molecule; a second container containing primers useful for amplifying said nucleic acid molecule; and instructions for using the probe and primers for facilitating the diagnosis of disease. Such a kit may further comprise a third container holding an agent for digesting unhybridised RNA.

According to a still further aspect of the invention, there is provided a transgenic or knockout non-human animal that has been transformed to express higher, lower or absent levels of a non-viral mutant retroelement nucleic acid molecule. Such transgenic animals will be invaluable in the study of TSE disease, and in the development of compounds that are effective in the diagnosis and treatment of these diseases. For example, the invention includes a method for screening for a compound effective to treat a TSE disease, by contacting a non-human transgenic animal that has been transformed to express higher, lower or absent levels of a non-viral mutant retroelement nucleic acid molecule with a candidate compound and determining the effect of the compound on the disease of the animal.

The invention will now be described by way of example, with reference to a model of disease caused by SINE retroelements.

### BRIEF DESCRIPTION OF THE FIGURE

FIG. 1 shows a scheme of TSE disease infection and progression.

### EXAMPLE 1

### Model of TSE Disease

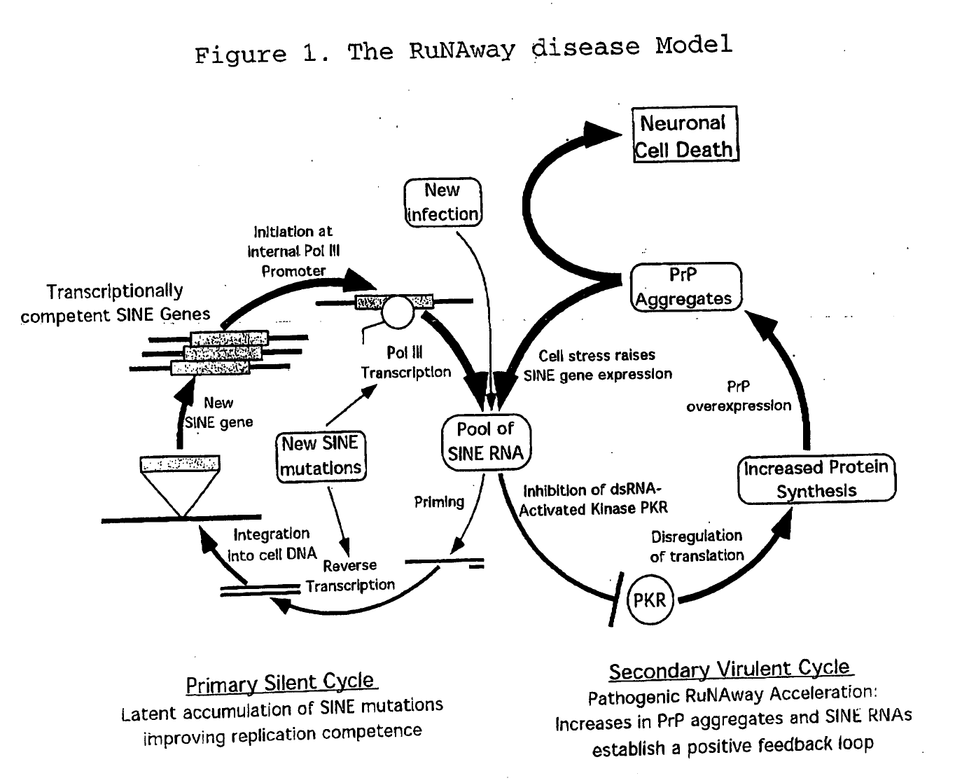

The disease scheme is depicted in FIG. 1.

In the primary cycle shown on the left, a clone of SINE genes gradually escapes from the default state of heavily suppressed replication. The error-prone reverse transcriptase provides a source of mutations and those that improve replication competence are iteratively incorporated into new genes.

As the SINE genes proliferate, the SINE RNA concentration increases, which may be aided by mutations that hinder RNA turnover Eventually SINE RNAs begin to titrate out the cellular pool of PKR, so causing the initiation of the secondary virulent cycle shown on the right side of FIG. 1. PrP overexpression leads to PrP deposits that stress the cell. In turn, this stress induces massive SINE transcription, ensuring that PKR remains fully inhibited.

The two cycles of the outlined scheme fit well with the observed TSE progression, with the primary silent cycle corresponding to the latent period of infection followed by an abrupt transition to the second virulent cycle coinciding with the onset of pathological disease symptoms. SINE elements have the required characteristics of the transmissible agent: they are small, can replicate in somatic cells, and, being solely composed of nucleic acid, will exhibit reproducible genetic behaviour, but will not elicit an immune reaction when infected into a new host.

The simple scheme described here is testable and can provide a framework for cell fractionation approaches aimed at isolating the transmissible agents in TSE disease. The scheme predicts that transfer of a TSE between species will lead to copies of a repetitive element present in the infected source material being multiply inserted into the genomes of newly infected cells and then being expressed as cellular RNA. Verification of this prediction should be technically straightforward.

### EXAMPLE 2

### Testing the Model

The availability of powerful techniques of molecular biology ensure that validation of the approach proposed herein will be relatively straightforward for an experimental lab with access to TSE material. Ideally, any validation approach will use scrapie, which is not known to be dangerous to humans, although equivalent approaches using other diseases may also be pursued.

Experiments fall into two classes: (1) Correlation with infectivity and (2) Demonstration of infectivity. In contrast to earlier attempts to isolate an unknown viral, viroid or virino nucleic acid agent (see, for example, Akowitz, A. et al (1994) NAR, 22, 1101-1107), this model very precisely defines what needs to be looked for.

1) Correlation

If the scrapie agent is a SE, transmission from sheep to hamster/mouse will result in proliferation of the SINE gene and RNA in the new host. Assuming that scrapie is indeed a mutated sheep SINE, the known sequence classes can be used to probe for the SINE transfer. These experiments can be done very easily, using existing infected material, and the validation experiment should be possible in weed.

If, however, scrapie was introduced to sheep from another source, the SINE sequence will not be so closely related to the sheep SINEs and it will take longer to find the precise causative agent.

Looking at genomic DNA, new insertions of the SINE genes will be random in each cell. To examine infected genomes by southern blotting, internal restriction sites will be needed to show that a clear cut band of a given size had been introduced with infection. Either internal restriction sites may be chosen from the known SD sequences, or a panel of frequent cutting (4-base targets) restriction enzymes may be used to guarantee that short internal fragments are generated. Alternatively, a clonal population of a persistently infected cell line such as the mouse SMB cell line (Rudyk, H. et al. (2000) J. Gen. Virol., 81, 1155-1164) will all have equivalent SINE gene insertions so that southern blotting would detect these as a series of bands absent from a control cell line.

2) Demonstration of Infecivity

Showing that a nucleic acid is transferred with the disease is an association only (though a useful one), and not a demonstration of infectivity. Experiments to demonstrate the latter connection will take longer as they will include the time Deeded for the disease to run its course after inoculation.

One approach will be to fractionate purified RNA on denaturing and non-denaturing gels, cut the gel up, extract nucleic acids from different migration points and inject into host animals. Peak infectivity will indicate where the infectious RNA species resided.

Once a correlation has been established between an RNA molecule and infectivity, that RNA molecule can be expressed recombinantly, for example, from a gene construct cloned into E. coli, so guaranteeing its purity. The RNA molecule can then be injected into susceptible animals, to demonstrate that infectivity resides in that RNA species alone. Alternatively, solid phase chemical synthesis of the full length RNA would provide an equally convincing demonstration.

### EXAMPLE 3

### Testing for TSE Infection

Once the precise nucleic acid agents for TSEs are known, tests that are (a) fast, and (b) sensitive can be applied before any pathological symptom is apparent. Current tests for PrP deposits can only detect animals that have been infected for years: this is a major limitation. The ability to test for the nucleic acids underlying TSEs is an extremely important application that flows from this model. Millions of tests will need to be performed until TSE diseases are under control or eradicated.

Nucleic acid tests could take advantage of PCR amplification and nucleic acid sequence determination. The tests would need to distinguish between the non-destructive indigenous SINE nucleic acid, and the mutated disease agent. As there may only be a few mutations in the disease agent relative to the harmless sequences, a sequencing step may usually be unavoidable.

Ideally, a PCR or hybridisation test should use oligonucleotide primers that distinguish between the isoforms.

### EXAMPLE 4

### Therapeutic Intervention in TSEs

In the longer term, knowledge of the transmissible agent for TSEs may allow the development of therapeutic approaches to hinder progression of the diseases.

The model of the invention suggests various intervention points for SINE RNA, PKR kinase, protein translation and PrP deposits. In particular, the demonstration that immunisation with Alzheimer's protein deposits protects mice from the disease (Schenk, D. et al. (1999) Nature, 400, 173-177) also suggests that PrP deposits may be cleared by immunisation.

1) RNAi Intervention

In many eukaryotes, including plants and nematode worms and flies, the presence of dsRNA will cause a homologous gene to be shut down. This phenomenon is termed gene silencing. The mechanism is not presently clear, though under intensive investigation, but inserting the dsRNA into worms and flies is remarkably easy. So far, it has been difficult to show this effect clearly in mammals, yet it seems to be a very generic property of eukaryotes, probably involved in anti-viral or anti-transposon defence.

Provided that such a mechanism can be invoked in mammals (and humans in particular) RNAi may be used to silence rogue retroelements, so terminating the proliferative cycle. This approach will require caution, since it is possible that SINEs have positive effects on the host genome. SINEs have coexisted with their hosts for 100s of millions of years and therefore could have acquired some symbiotic functions with the host cells.

2) PKR and control of Translation

Experimental approaches to protein targeting now indicate that it may be possible to introduce modified proteins into somatic cells for therapeutic purposes.

The PKR kinase is a very powerful regulator of translation that is central lo viral defence and likely has other inputs too One approach to therapeutic intervention for TSEs may be to introduce an engineered PKR into the animal cells, so that the protein can no longer be inhibited by SINEs and therefore downregulate translation in the infected cells. This will lead to reduced PrP expression and prevention of PrP deposits.

1. A method for diagnosing an animal for a transmissible spongiform encephalopathy disease, said method comprising testing the animal for the presence of mutant non-viral retroelement nucleic acid molecules, wherein the presence of said mutant retroelement nucleic acid molecules is indicative of infection.

2. A method according to claim 1, wherein said testing comprises the steps of:
a) contacting a sample of tissue from the animal with a nucleic acid probe under stringent conditions that allow the formation of a hybrid complex between the mutant non-viral retroelement nucleic acid molecule and the probe;
b) contacting a control sample with said probe under the same conditions used in step a); and
c) detecting the presence of hybrid complexes in said samples;
wherein detection of levels of the hybrid complex in the animal sample that differ from levels of the hybrid complex in the control sample is indicative of the TSE disease.

3. A method according to claim 2, wherein said probe is immobilised on a support, such as on a nucleic acid array.

4. A method according to claim 1, wherein said testing comprises the steps of:
a) contacting a sample of nucleic acid from tissue of the animal with a nucleic acid primer under stringent conditions that allow the formation of a hybrid complex between the non-viral retroelement nucleic acid molecule and the primer;
b) contacting a control sample with said primer under the same conditions used in step a);
c) amplifying the sampled nucleic acid; and
d) detecting the amplified nucleic acid from both patient and control samples;
wherein detection of amplified nucleic acid in the animal sample that differ significantly from the amplified nucleic acid in the control sample is indicative of TSE disease.

5. A method according to claim 4, wherein said detection step d) comprises detecting the level of amplified nucleic acid from both patient and control samples, and wherein detection of levels of the amplified nucleic acid in the animal sample that differ significantly from levels of the amplified nucleic acid in the control sample is indicative of TSE disease.

6. A method according to claim 4, wherein said detection step d) comprises the step of sequencing the amplified nucleic acid from both patient and control samples, wherein detection of amplified nucleic acid in the animal sample of a different sequence to that of the amplified nucleic acid in the control sample is indicative of TSE disease.

7. A method according to claim 1, wherein said method comprises the steps of:
a) obtaining a tissue sample from an animal being tested for TSE disease;
b) isolating nucleic acid from said tissue sample; and
c) diagnosing the animal for disease by detecting the presence of mutant non-viral retroelement nucleic acid molecule as an indication of the TSE disease.

8. A method according to claim 7, additionally comprising the step of analysing the sequence of the mutant non-viral retroelement nucleic acid molecule.

9. A method according to any one of the preceding claims, that is carried out in vitro.

10. A method of treating a TSE disease in an animal in need of such treatment by administering to an animal a therapeutically-effective amount of a compound that is effective to counteract the effect of a non-viral mutant retroelement nucleic acid molecule in inducing TSE disease.

11. A method according to claim 10, wherein said compound is an RNAi molecule that is targeted to the non-viral mutant retroelement nucleic acid molecule.

12. A method according to claim 11, wherein said compound is a modified PKR molecule, the activity of which is not inhibited by a non-viral mutant retroelement nucleic acid molecule.

13. A method according to claim 12, wherein said modified PKR molecule is administered directly to the animal.

14. A method according to any one of claims 10-12, wherein a nucleic acid molecule encoding said compound is administered directly to the animal in an expressible vector, wherein said vector comprises expression control sequences operably linked to the nucleic acid molecule.

15. A method of monitoring the therapeutic treatment of a TSE disease in an animal, comprising monitoring over a period of time the level of expression of a non-viral mutant retroelement nucleic acid molecule in tissue from said patient, wherein altering said level of expression over the period of time towards a control level is indicative of regression of said disease.

16. A method according to any one of the preceding claims, wherein said non-viral mutant retroelement nucleic acid molecule is a small interspersed nuclear element (SINE), or a long interspersed nuclear element (LINE).

17. A method according to any one of the preceding claims, wherein said transmissible spongiform encephalopathy disease is Creutzfeldt-Jakob disease (CJD), new variant Creutzfeldt-Jakob disease (vCJD), Kuru, fatal familial insomnia (FFI) or Gerstmann-Straussler syndrome (GSS) in humans, scrapie in sheep, bovine spongiform encephalopathy (BSE), feline spongiform encephalopathy (FSE), transmissible mink encephalopathy (TME), or chronic wasting disease (CWD) in undomesticated mule deer and elk.

18. A method according to any one of the preceding claims, wherein said animal is a mammal.

19. A method according to claim 18, wherein said animal is a domestic ungulate.

20. A non-viral mutant retroelement nucleic acid molecule that is implicated in the cause or progression of a TSE disease.

21. A nucleic acid molecule according to claim 20, wherein said non-viral mutant retroelement nucleic acid molecule is a mutant SINE, or a mutant LINE nucleic acid molecule.

22. A method for the identification of a compound that is effective in the treatment and/or diagnosis of TSE disease, comprising contacting a non-viral mutant retroelement nucleic acid molecule with one or more compounds suspected of possessing affinity for said non-viral mutant retroelement nucleic acid molecule, and selecting a compound that binds specifically to said non-viral mutant retroelement nucleic acid molecule.

23. A compound identifiable by a method according to claim 22.

24. A compound according to claim 23, which is a nucleic acid molecule, an enzyme, a small organic molecule, a peptidomimetic, an inorganic molecule, a peptide, a polypeptide or an antibody.

25. A compound according to claim 24, wherein said compound is a RNAi molecule that is targeted to the non-viral mutant retroelement nucleic acid molecule implicated in TSE disease.

26. A compound according to claim 24, wherein said compound is a modified PKR molecule, that is not inhibited by non-viral mutant retroelement nucleic acid molecules.

27. A kit useful for diagnosing TSE disease comprising a first container containing a nucleic acid probe that hybridises under stringent conditions with a non-viral mutant retroelement nucleic acid molecule; a second container containing primers useful for amplifying said nucleic acid molecule; and instructions for using the probe and primers for facilitating the diagnosis of disease.

28. The kit of claim 27, further comprising a third container holding an agent for digesting unhybridised RNA.

29. A transgenic or knockout non-human animal that has been transformed to express higher, lower or absent levels of a non-viral mutant retroelement nucleic acid molecule.

30. A method for screening for a compound effective to treat a TSE disease, by contacting a non-human transgenic animal according to claim 29 with a candidate compound and determining the effect of the compound on the TSE disease of the animal.
